(19) United States
(12) Patent Application Publication
(10) Pub. No.: US 2002/0137118 A1
(43) Pub. Date: Sep. 26, 2002

(54) Biologically active protein folding intermediates

(75) Inventors: Masayori Inouye, Piscataway, NJ (US); Ujwal Shinde, Gladstone, OR (US); Xuan Fu, Piscataway, NJ (US)

Correspondence Address: LYON & LYON LLP, 633 WEST FIFTH STREET, SUITE 4700, LOS ANGELES, CA, 90071, US

(73) Assignee: UNIVERSITY OF MEDICINE AND DENTISTRY OF NEW JERSEY

(21) Appl. No.: 09/935,744

(22) Filed: Aug. 24, 2001

Related U.S. Application Data
(60) Provisional application No. 60/227,468, filed on Aug. 24, 2000.

(51) Int. Cl. C12Q 1/37; C12P 21/06

(52) U.S. Cl. 435/23; 435/68.1

(57) ABSTRACT

This invention provides biologically active protein folding intermediates and methods of making and using the same.

This application claims the benefit of U.S. Provisional Application No. 60/227,468 filed on Aug. 24, 2000.

Work described herein was supported by NIH grant GM-56419-03.

### BACKGROUND OF THE INVENTION

Proteins fold rapidly and they often do so through distinct folding intermediates. Baldwin, R. L. et al., Trends Biochem. Sci. 24, 26-33 (1999). Although data suggest that such intermediates promote protein folding (Baldwin, R. L. et al. (1999), supra; Baldwin, R. L. et al., Trends Biochem. Sci. 24, 77-83 (1999)), evidence to the contrary indicates that escape from the accumulation of the intermediates accelerates folding rates (Hagihara, Y. et al., Molecular Chaperones in the Life Cycles of Proteins (Fink A L and Goto Y eds) 1-33, Marcel Decker, NY (1998); Creighton, T. E., Trends Biochem. Sci. 22, 6-10 (1997)).

While molecular chaperones prolyl-peptidyl isomerases and disulfide isomerases can facilitate folding (Ellis, R. J., Curr. Biol. 9, 352-355 (1999)), certain proteins require that their N-terminal extensions function as intramolecular chaperones (IMCs) to assist folding (Shinde, U. P. et al., Sem. Cell Dev. Biol. 11, 35-44 (2000); Inouye, M., Enzyme 45, 314-321 (1991)). Upon completion of folding, the IMC is removed through autolytic or exogenous proteolytic activity. Ikemura, H. et al., J. Biol. Chem. 262, 7859-7864 (1987).

Subtilisin (Ikemura, H. et al., supra; Shinde, U. P. et al., Nature 389, 520-522 (1997); Zhu, X. et al., Nature 339, 483-484 (1989); Shinde, U. P. et al., J. Biol. Chem. 274, 15615-15621 (1999); Ruan, B. et al., Biochemistry 38, 8562-8571 (1999); Wang, L. et al., Biochemistry 37, 3165-3171 (1998)), α-lytic protease (Silen, J. L. et al., Nature 341, 462-464 (1989); Baker, D. et al., Nature 356, 263-265 (1992); Sohl, J. L. et al., Nature 395, 817-819 (1998); Winther, J. R. et al., Prac. Natl. Acad. Sci. USA 88, 9330-9334 (1991)), and carboxypeptidase Y (Winther, J. R. et al., supra) constitute the best studied examples of this widely emerging class of proteins (Shinde, U. P. (2000), supra). Although subtilisin (Eder, J. et al., Biochemistry 32, 18-26 (1993); Eder, J. et al. J. Mol. Biol. 233, 293-304 (1993); Shinde, U. P. et al., J. Mol. Biol. 252, 25-30 (1995)) and α-lytic protease (Baker, D. et al., supra; Sohl, J. L. et al., supra) have been shown to fold through distinct intermediates, the exact mechanisms of IMC-mediated folding remain elusive.

### SUMMARY OF THE INVENTION

This invention provides biologically active protein folding intermediates and methods of making and using the same.

One aspect of this invention relates to a method for preparing a biologically active intermediate of a folding protein comprising contacting an intermediate of a folding protein with an effective amount of a substrate or reducing agent. Another aspect of this invention relates to a biologically active intermediate prepared by such method.

A further aspect of this invention relates to a drug design assay comprising assessing a drug candidate's ability to stabilize or prevent formation of a biologically active intermediate of a folding protein.

### BRIEF DESCRIPTION OF THE DRAWINGS

FIG. 1A is an X-ray crystallography image of the IMC-subtilisin complex. Jain, S. C. et al., J. Mol. Biol. 284, 137-144 (1998). The C-terminal part of the IMC domain interacts with the substrate binding domain. The effects of a M(−60)T substitution can be suppressed by an Ser 188 to Leu (S188L) mutatation 47 Å away. Kobayashi, T. et al., J. Mol. Biol. 226, 931-933 (1992).

FIG. 1B is a graph plotting the folding kinetics of precursors wild type IMC-subtilisin, M(−60)C-IMC-subtilisin, IMC-S188C-subtilisin and M(−60)C-IMC-S188C-subtilisin (2Cys-IMC-subtilisin). The percent folded was calculated as % F=[(A−A_{D})/(A_{F}−A_{D})]×102, where A represents ellipticity at 225 nm at a given time, and AF and AD represent the signals of fully folded and completely denatured precursors, respectively. Folding rate constants for wild type IMC-subtilisin, M(−60)C-IMC-subtilisin, IMC-S188C-subtilisin and 2Cys-IMC-subtilisin are 0.058, 0.044, 0.049 and 0.043 sec⁻¹, respectively.

FIG. 1C is a circular dichroism (CD) spectroscopy image of the maturation of rapidly diluted precursors. The gels show the processing of folded precursors (lanes 1, 4, 6 and 8) to mature proteases (lanes 2, 5, 7 and 9) upon incubation at 4° C. for 12 hours.

FIG. 1D is a graph plotting the CD spectra of mature proteins 12 hours after refolding of precursors (FIG. 1C, lanes 2, 5, 7 and 9).

FIG. 2A is a nonreducing SDS-PAGE analysis of 2Cys-IMC-subtilisin. The uncrosslinked 2Cys-IMC-subtilisin (shown by “Re” marker in lanes 1 and 4) when oxidized during folding underwent ˜50% intramolecular crossing (shown by “Ox” marker in lane 2). Very little intermolecular crosslinking (lane 2, shown by “Di” marker) occurred. Purified stable crosslinked intermediate conformer (CLIC) (lane 3), when incubated with 1 mM dithiothreitol (DTT), underwent reduction (lane 4).

FIG. 2B is a graph plotting the CD spectra of the secondary structures of rapidly diluted precursors. The 2Cys-IMC-subtilisin was unstructured in 4 M urea. Under reducing conditions and in the absence of a denaturant, wild type IMC-subtilisin and uncrosslinked 2Cys-IMC-subtilisin adopt similar secondary structures but display differences from CLIC under nonreducing conditions. However, if folded CLIC is reduced by DTT, it acquires a conformation similar to that of 2Cys-IMC-subtilisin under reducing conditions (FIG. 2D); the spectrum is not identical to that of 2Cys-IMC-subtilisin because the sample contained both crosslinked and uncrosslinked CLIC (FIG. 2D, lane 3). After an overnight incubation under reducing conditions, CLIC completely matured into active S188C-subtilisin (FIG. 2C, inset, lane 2). FIG. 2B (inset) is a graph plotting the kinetic traces of CLIC and uncrosslinked 2Cys-IMC-subtilisin compared to wild type IMC-subtilisin. The rate-constants for CLIC is 0.29 s⁻¹ which is >˜6-fold that of the uncrosslinked protein.

FIG. 2C is a graph plotting the tryptophan fluorescence spectra of CLIC and uncrosslinked 2Cys-IMC-subtilisin. FIG. 2C (inset) is a CD spectroscopy image of the maturation of uncrosslinked 2Cys-IMC-subtilisin (lanes 1 and 2) 24 hours after rapid dilution into refolding buffer with DTT. However, CLIC (lanes 4 and 5) after the same time interval does not undergo maturation in the absence of DTT. Lane 3 represents the molecular weight marker.

FIG. 2D is a CD spectroscopy image of the effects of different conditions on CLIC. CLIC was stable for several weeks (lane 1) in the absence of any reducing agent. However, when CLIC was incubated with 1 mM DTT it underwent autoprocessing in ˜1 hour (lane 3). Lane 2 represents CLIC after activation with a small peptide substrate.

FIG. 3A is a graph plotting the activation time of mature S188C-subtilisin (filled circles) and CLIC (open circles) incubated with a peptide N-succinyl-Ala-Ala-Pro-Phe-p-nitroanilide at 25° C. in an assay buffer. The activation time was determined by extrapolating the fast phase to y=0. FIG. 3A (inset) depicts the analytical gel filtration profiles for CLIC and activated crosslinked intermediate conformer (A-CLIC).

FIG. 3B is a graph plotting the change in activation time of CLIC as a function of N-succinyl-Ala-Ala-Pro-Phe-p-nitroanilide (filled squares) and N-succinyl-Ala-Ala-Pro-Leu-p-nitroanilide (filled triangles) concentration.

FIG. 3C is a CD spectroscopy image of the effects of mature S188C-subtilisin (top panel, under reducing conditions) and CLIC (bottom panel, under oxidizing conditions) on a large protein substrate, bovine serum albumin (BSA).

FIG. 3D is a schematic representation of intermediate activation by a small peptide substrate. A disulfide bond between residues Met (−60) and Ser 188 traps the IMC and the protease domain. Large substrates may not bind to CLIC probably due to steric hindrance.

### DETAILED DESCRIPTION OF THE INVENTION

I. Overview

The 77-residue subtilisin propeptide functions as an intramolecular chaperone (IMC) that facilitates the folding of its protease domain through an experimentally detectable folding intermediate. Eder, J. et al., Biochemistry 32, 18-26 (1993); Eder, J. et al., J. Mol. Biol. 233, 293-304 (1993); Shinde, U. P. et al. (1995), supra. While similar intermediates exist in several protein-folding models (Booth, D. R. et al., Nature 385, 787-793 (1997); Jamin, M. et al., Nature Struct. Biol. 3, 613-8 (1996); Schultz, C. P. Nature Struct. Biol. 7, 7-10 (2000)), their physiological relevance and importance in the folding kinetics remain contested (Hagihara, Y. et al., supra).

The intramolecular chaperone (IMC) of subtilisin was used to trap a partially folded, stable crosslinked intermediate conformer (CLIC) through a disulfide bond between mutated IMC and subtilisin. The trapped CLIC contains non-native interactions. The inventors have discovered that CLIC could be induced into a catalytically active form by incubating it with small peptide substrates. The structure and catalytic properties of the activated crosslinked intermediate conformer (A-CLIC) differ from those of the fully folded enzyme in that A-CLIC lacks any endopeptidase activity toward a large protein substrate. The results show that a disulfide-linked partially folded protein can be induced to acquire catalytic activity with a substrate specificity that is different from completely folded subtilisin. The results also suggest that protein folding intermediates may perform biological functions and play crucial roles in correcting and altering protein folding.

II. Definitions

“Protein folding” refers to the process by which polypeptide chains assume their three-dimensional conformation, either during normal protein biosynthesis or experimental protein reconstitution.

“Denaturation” refers to the loss of native structure of a biomolecule, which could result in the loss of function.

“Domain” refers to an independently folded unit within a protein, often joined by a flexible segment of the polypeptide chain.

“Molecular chaperones” refer to a family of cellular proteins that mediate the correct assembly or disassembly of other polypeptides, and in some cases their assembly into oligomeric structures, but which are not components of those final structures. It is believed that chaperones assist polypeptides to self-assemble by inhibiting alternative assembly pathways that produce nonfunctional structures. Examples of classes of molecular chaperones include, without limitation, nucleoplasmins, chaperonins and heat-shock proteins.

“Protein conformation” refers to the characteristic 3-dimensional shape of a protein, including the secondary, supersecondary (motifs), tertiary (domains) and quaternary structure of the peptide chain.

“Secondary structure” refers to the conformational arrangement (αa-helix, β-pleated sheet, etc.) of the backbone segments of a macromolecule, such as a polypeptide chain of a protein, without regard to the conformation of the side chains or the relationship to other segments.

III. Methods for Preparing Biologically Active Protein Folding Intermediates

This invention provides a method for preparing a biologically active intermediate of a folding protein comprising contacting an intermediate of a folding protein with an effective amount of a substrate or reducing agent.

Examples of a folding protein include, without limitation, subtilisin, α-lytic protease and carboxypeptidase Y.

The specific reducing agent or substrate and the amount of the reducing agent or substrate may vary depending upon the particular folding protein and other factors well known in the art. Examples of a reducing agent include, without limitation, glutathione, cysteine, cystamine, thioglycollate, dithioerythritol, dithiothreitol, dithioerythritol and mercaptoethanol.

This invention also provides a biologically active intermediate prepared by a method of the invention.

IV. Drug Design Assays

This invention further provides a drug design assay comprising assessing a drug candidate's ability to stabilize or prevent formation of a biologically active intermediate of a folding protein. In one embodiment, the biologically active intermediate is prepared by contacting an intermediate of a folding protein with an effective amount of a substrate or reducing agent.

### EXAMPLES

The following examples are illustrative of the present invention and are not intended to be limitations thereon.

### Example 1

### Substrate-Induced Activation of a Trapped IMC-Mediated Protein Folding Intermediate Purification of CLIC

Proteins were expressed as inclusion bodies (Fu, X. et al., J. Biol. Chem. 275, 16871-16878 (2000)) containing both the crosslinked and uncrosslinked forms of M(−60)C-IMC-S188C-subtilisin (2Cys-IMC-subtilisin). Separation of the precursor forms was achieved by using a preparative gel electrophoresis apparatus Model 591-B (BioRad). Electrophoresis was carried out under nonreducing conditions using an acid-urea polyacrylamide gel as described. Panyim, S. et al., Arch. Biochem. Biophys. 130, 337-346 (1969).

Folding of Denatured Precursors

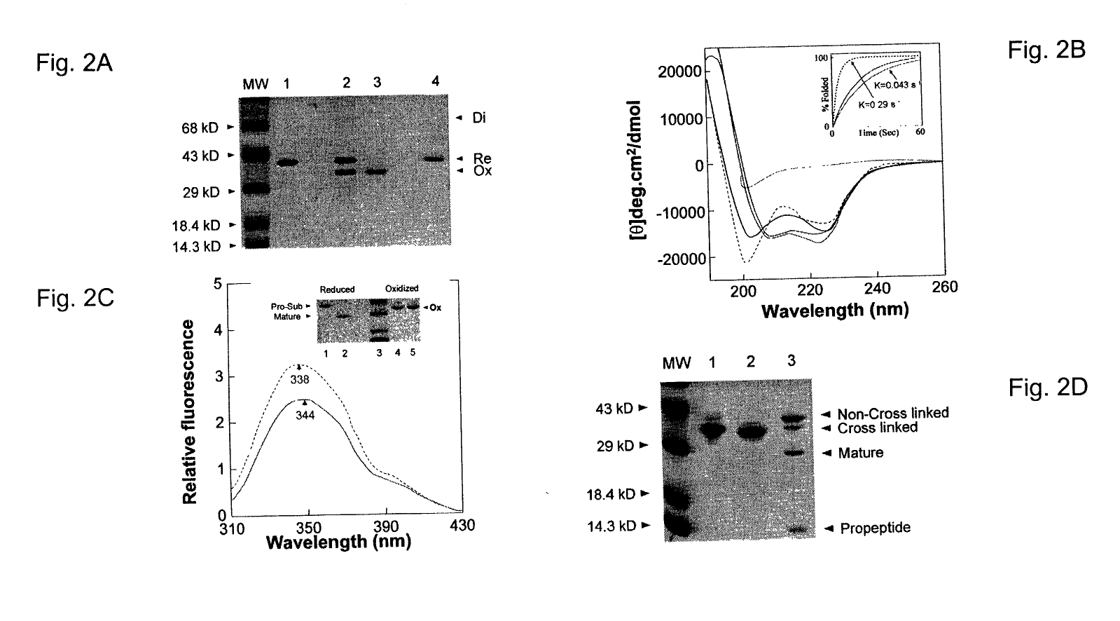

Denatured precursor proteins in 6 M GdnHCl, pH 4.8 (1.25 mg ml⁻¹) were used for refolding. The temperature for renaturation was maintained at 4° C. and folding initiated through rapid dilution of 200 μl of the protein into 2800 μl of the refolding buffer (50 mM Tris-HCl, pH 7.0, containing 0.5 M (NH₄)₂SO₄, 1 mM CaCl₂). When refolding was carried out under reducing conditions, the denatured protein and the refolding buffer contained 5 mM DTT. The kinetics of folding were monitored by rapidly diluting the denatured precursors (15-fold dilution) into a quartz cuvette (1 cm path length) and simultaneously recording the changes in the CD spectra at 225 nm. Data were collected at 1 second time intervals and represent the average of three independent experiments. By using nonlinear regression, the traces were found to fit a single exponential rate constant. The Prism Graph-pad software Version 2.01 was used for the data fitting analysis and graph plotting. After completion of the folding experiments, aliquots of the sample were removed. Each aliquot was used separately for assaying proteolytic activity and for determining the extent of precursor maturation (trichloroacetic acid precipitation (TCA) followed by a nonreducing SDS-PAGE). The data show that, similar to the wild type precursor (Fu, X. et al, supra), the 2Cys-IMC-subtilisin precursor and CLIC showed no changes in their CD spectra, and >99% of the refolded proteins remained as precursors after 1,500 seconds of refolding (FIG. 2B). Folded CLIC remained stably trapped for several weeks. Folding through a stepwise dialysis when required was carried out as described (Fu, X. et al, supra).

Oxidization of 2Cys-IMC-Subtilisin

The protein refolded using stepwise dialysis was oxidized by bubbling air in the refolding buffer (50 mM Tris-HCl, pH 7.0, 0.5 M (NH₄)₂SO4, 1 mM CaCl₂ and 4 M urea) around the dialysis tubing. The oxidization reaction to promote disulfide bond formation was carried out for 24 hours with four changes with fresh refolding buffer.

Enzymatic Activity

An aliquot of the sample is incubated at 25° C. in 200 μl of 50 mM Tris-HCl, pH 8.5, containing 1 mM CaCl₂. The enzymatic activity of subtilisin was estimated by monitoring the release of p-nitroaniline through changes in absorbance at 405 nm using a BioRad UV-microplate reader as described (Fu, X. et al, supra). The activation time for CLIC was calculated as the time of transition from the slow to the fast phase. Activity against bovine serum albumin (BSA) was estimated as follows. Uncrosslinked 2Cys-IMC-subtilisin and CLIC (10 μg each) were incubated at 4° C. with BSA (600 μg) in 300 μl of 50 mM Tris-HCl, pH 7.0, containing 0.5 M (NH₄)₂SO4 and 1 mM CaCl₂. Aliquots (25 μl) of the mixture were removed and precipitated using 3 μl of 100% TCA. The precipitate was initially washed with 3% TCA followed by a wash with 100% acetone. The dried pellet was resuspended in the SDS loading dye with or without DTT and subjected to SDS-PAGE.

Biophysical Studies

CD measurements were performed on an automated AVIV 60DS spectrophotometer maintained at 4° C. and spectra were measured between 260 and 190 nm as described (Shinde, U. P. et al. (1997), supra). The protein concentration was 0.3 mg ml⁻¹ and a path length of 0.1 cm was used. Spectra were analyzed using a nonconstrained least-square analysis (Greenfield, N. J., Anal. Biochem. 235, 1-10 (1996)) using the spectra of polypeptide models as defined by Brahms and Brahms (Brahms, S. et al., J. Mol. Biol. 138, 149-178 (1980). In thermal denaturing measurements, temperatures were increased from 10-90° C. at a 0.5° C. interval, equilibrated for 6 seconds at each temperature. Fluorescence studies were carried out on an SLM Aminco Bowman Series 2 luminescence spectrometer at 4° C. For the intrinsic fluorescence experiments, the proteins were excited at 295 nm and emission scans recorded at 300-410 nm.

Analytical gel Filtration Chromatography

Gel filtration was carried out on an AKTA-FPLC using a Superdex-peptide 3.2/30 column (Amersham-Pharmacia Biotech). Protein samples (25 μl) were loaded on the column and eluted using 50 mM Tris-HCl₁, pH 7.0, containing 0.5 M (NH₄)₂SO4 using a flow rate of 50 μl min⁻¹.

Results

Ser 188 and Met (−60) interact during folding

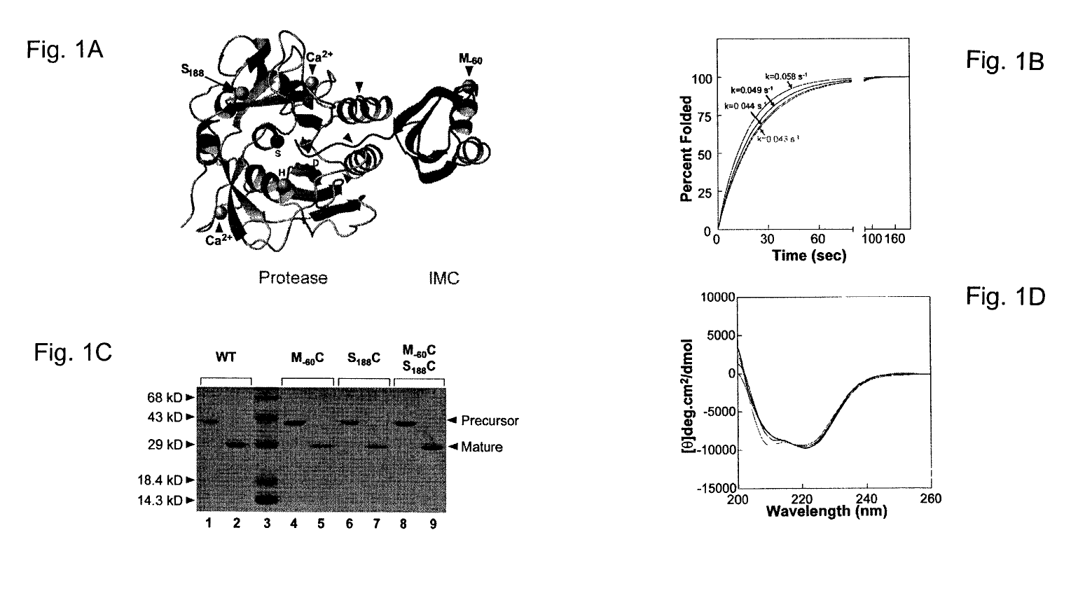

An in viva protease assay demonstrated that the dramatic reduction in subtilisin activity by the mutation of Met (−60) to Thr (M(−60)T) within the IMC domain could be overcome by an S188L substitution (S188L) in the protease domain. The number indicates the distance of the residue from the cleavage site while the negative sign indicates that the residue is at the N-terminus of the cleavage site. Hence, Met −60 indicates that this residue is located 60 residues of the N-terminus of the cleavage site. Because X-ray crystallography of the cleaved IMC in complex with mature subtilisin (Gallagher, T. et al., Structure 3, 907-914(1995); Jain, S. C. et al., supra) showed these two residues to be 47 Å apart (FIG. 1A), Met (−60) in the IMC was suggested to interact with Ser 188 within mature subtilisin during folding (Inouye, M., supra). To explore whether these residues are in close proximity during subtilisin folding, the inventors attempted to trap this interaction through a covalent bond.

Because the primary sequence of subtilisin does not contain any Cys residue (Inouye, M., supra), site-directed mutagenesis was used to introduce an M(−60)C mutation in the IMC and S188C substitution within subtilisin. To examine the effects of individual mutations on folding, mutant and wild type IMC-subtilisin precursors were renatured under reducing conditions and their folding their folding kinetics monitored using circular dichroism (CD) spectroscopy (Fu, X. et al., supra; see EXAMPLE 1). The folding kinetics of the single Cys mutants (M(−60)C-IMC-subtilisin and IMC-S188C-subtilisin) and the double Cys mutant protein, 2Cys-IMC-subtilisin under reducing conditions can be described by a single-exponential equation similar to that of wild type IMC-subtilisin. The mutant precursors, however, folded slightly slower than wild type IMC-subtilisin (FIG. 1B). All the refolded precursors could autoprocess and degrade their IMC domains to yield mature proteins (FIG. 1C) with secondary structures similar to those of wild type subtilisin (FIG. 1D), indicating that the Cys substitutions do not significantly alter precursor maturation under reducing conditions.

Trapping of a CLIC by Cys Oxidation

Denatured 2Cys-IMC-subtilisin (FIG. 2A, lane 1) was refolded and oxidized to facilitate disulfide bond formation (see EXAMPLE 1). The SDS-PAGE experiment shown in FIG. 2A was carried out under nonreducing conditions and, therefore, the oxidized protein shows two bands (FIG. 2A, lane 2). The 40 kDa and 35 kDa bands (shown by markers “Re” and “Ox”, respectively, in FIG. 2A) correspond to the uncrosslinked 2Cys-IMC-subtilisin and CLIC, respectively. Lowering the concentration of urea to 2 M prior to this reaction improved the yield of the 35 kDa band (data not shown) and suggests that the interaction occurs during the productive folding pathway. Since the oxidation reaction showed ˜50% crosslinking efficiency (FIG. 2A, lane 2), the 35 kDa CLIC was separated from the 40 kDa uncrosslinked 2Cys-IMC-subtilisin using preparative gel electrophoresis that was carried out under nonreducing conditions (see EXAMPLE 1). When the purified 35 kDa CLIC (FIG. 2A, lane 3) was treated with a 1 mM DTT for 1 hour and loaded on a nonreducing SDS polyacrylamide gel, it underwent reduction and migrated as a 40 kDa protein (FIG. 2A, lane 4). This confirms that formation of an intramolecular disulfide bond alters the migration of CLIC. Furthermore, residual DTT from the sample in lane 4 did not diffuse into lane 3 and, therefore, did not affect the crosslinked protein in other lanes during electrophoresis. The 35 kDa protein was also identified as 2Cys-IMC-subtilisin by mass spectrometry (data not shown).

The precursor adopts no preferred secondary structure in 4 M urea (FIG. 2B), and very little of the protein undergoes intermolecular crosslinking (shown by marker “Di” in FIG. 2A, lane 2). Since Ser 188 and Met (−60) are 47 A apart (FIG. 1A) in the completely folded IMC-subtilisin (Gallagher, T. et al., supra; Jain, S. C. et al., supra), the results suggest that the intramolecular interaction between these residues in the trapped intermediate are non-native but highly specific. The formation of CLIC can also be observed when protein expression was under control of the T7-promoter (data not shown). CLIC can be separated from the uncrosslinked and intermolecular crosslinked forms using preparative gel electrophoresis (Panyim, S. et al., supra).

The folding rate constants and the kinetic traces of CLIC and uncrosslinked 2Cys-IMC-subtilisin were monitored as described (Fu, X. et al, supra). CLIC acquires secondary structure approximately ˜6-fold faster than the uncrosslinked precursor (FIG. 2B, inset). This suggests that the interactions between residues ˜60 and 188, which are trapped through an intramolecular disulfide bond, enhance the rate of folding of the precursor. The secondary structures of uncrosslinked 2Cys-IMC-subtilisin folded under reducing conditions and those of CLIC folded under nonreducing conditions are compared in FIG. 2B. Both precursors were refolded from their denatured using a rapid dilution folding method (Fu, X. et al, supra). The uncrosslinked 2Cys-mutant displays a secondary structure similar to tht of the wild type protein, but significantly different from that of CLIC upon refolding. The proteins depicted in FIG. 2B remained trapped as precursors during the time scale of the experiments in a manner similar to the wild type subilisin precursor (Fu, X. et al, supra). Upon incubation at 4° C. for 24 hours, the uncrosslinked 2Cys-IMC-subtilisin underwent maturation to give S188C-subtilisin (FIG. 2C, inset, lanes 1 and 2), which has a secondary structure similar to that of wild type subtilisin (FIG. 1D). However, CLIC remained trapped as a precursor and was stable for several weeks (FIG. 2C, inset, lanes 4 and 5). When compared with the uncrosslinked precursor, CLIC showed a shift in λₘₐₓ of the tryptophan fluorescence spectrum to a shorter wavelength (FIG. 2C), which suggests that CLIC and the uncrosslinked precursors differ in their conformations. Moreover, when compared with the uncrosslinked precursor, CLIC displayed a two-fold fluorescence increase in the presence of 100 μM 1-anilino-8-naphtalene sulfonic acid (data not shown), suggesting that the solvent accessible hydrophobic surface of CLIC increases (Shinde, U. P. et al. (1995), supra). Upon addition of a reducing agent, CLIC cleaved off its IMC domain to give mature subtilisin (FIG. 2D, lane 3). Moreover, reduction of the disulfide bond induced structural changes within CLIC, and its CD spectrum becomes similar to that of the wild type precursor (FIG. 2B). This indicates that CLIC has the potential to adopt a native-like conformation and undergoes maturation in the presence of a reducing agent.

Peptide-Induced Activation of CLIC

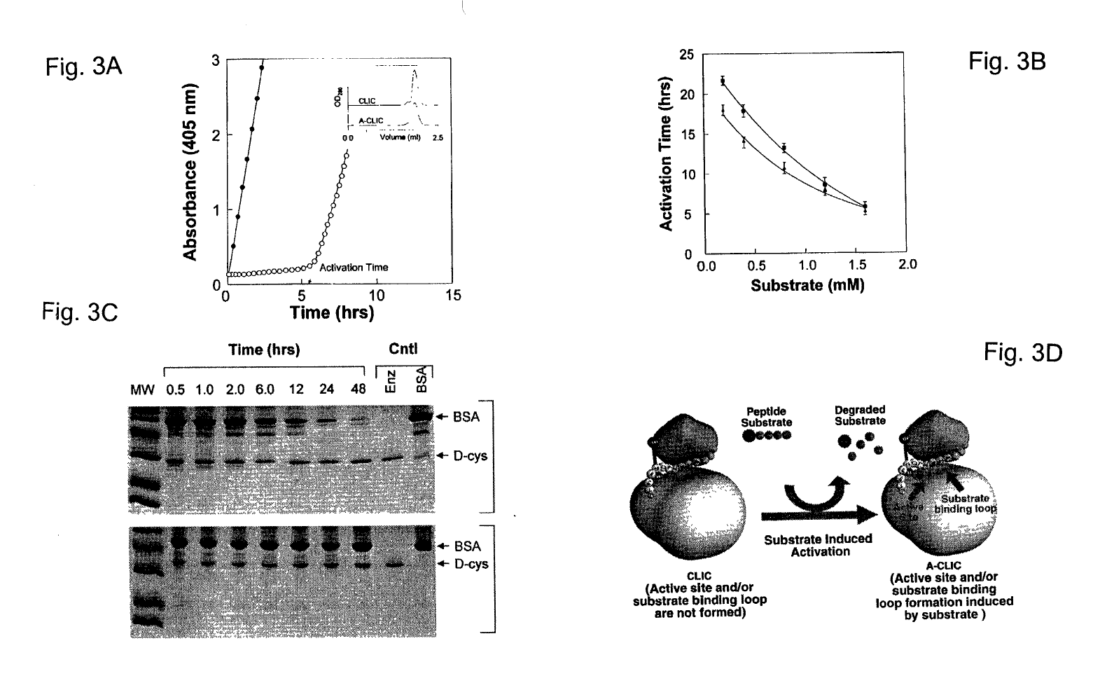

The IMC domain functions as a temporary inhibitor of subtilisin activity (Shinde, U. P. et al. (1997), supra; Zhu, X. et al., supra; Shinde, U. P. et al. (1999), supra). Thus, auto-proteolysis is necessary for subtilisin activity. Ikemura, H. et al., supra. Since trapping of CLIC through a disulfide bond forces the interactions of the IMC domain with subtilisin to remain non-native, the consequences of these interactions on a peptide substrate were examined. CLIC (FIG. 2C, inset, lane 4) was incubated with a synthetic substrate (N-succinyl-Ala-Ala-Pro-Phe-p-nitroanilide) in the absence of a reducing agent. In the case of CLIC, the release of p-nitroaniline (monitored using OD₄₀5) through substrate cleavage as a function of time is biphasic (FIG. 3A). The slow phase displayed a velocity ˜1% of the fast phase, which occured after a period of ˜5 hours of incubation with the substrate. The fast phase is due to substrate-induced activation of CLIC. The velocity of substrate degradation by the activated CLIC in the second phase was similar to that of mature S188C-subtilisin under identical conditions (FIG. 3A). Upon completion of the fast phase, CLIC remained trapped in a stable conformation (TABLE 1) for several weeks (FIG. 2D, lane 2) with insignificant changes in its CD spectrum (data not shown).

TABLE 1
COMPARISON OF THE STRUCTURAL PROPERTIES OF CLIC
WITH THE IMC-S188C-SUBTILISIN COMPLEX

| | | | | Uncrosslinked |
|---|---|---|---|---|
| | | IMC-S118C- | | 2Cys- |
| Property | Mature | subtilisin | CLIC | precursor |
| T_{M}¹ (° C.) | 58.5 | 53.2 | 52.4 | — |
| Structure | | | | |
| (%) | | | | |
| α-helix | 32 | 31 | 11 | 30 |
| β-sheet | 21 | 21 | 5 | 20 |
| β-turn | 9 | 10 | 7 | 9 |
| Random | 38 | 38 | 77 | 40 |
| coil | | | | |

¹Melting temperature

This suggests that the fast phase of the reaction does not occur due to IMC degradation or dramatic conformational changes. The precise mechanism of the nearly all-or-none activation observed for CLIC is presently unknown. One mechanism that could account for such a transition would require a multibody collision that results in a catalytically active aggregate that subsequently initiates the rest of the system. However, A-CLIC remains a monodisperse species, and its migration on a gel filtration column does not differ from that of CLIC (FIG. 3A, inset). Furthermore, the abrupt change in activity and the shape of the activation profile are not affected by changes in concentration of CLIC (data not shown).

Next the inventors examined the effects of the concentrations of two different synthetic substrates (N-succinyl-Ala-Ala-Pro-Phe-p-nitroanilide and N-succinyl-Ala-Ala-Pro-Leu-p-nitroanilide) on the activation time of CLIC (FIG. 3A). An increase in substrate concentration lowered the activation time of both peptide substrates (FIG. 3B) However, at lower concentrations, N-succinyl-Ala-Ala-Pro-Leu-p-nitroanilide appears to be a better activator than N-succinyl-Ala-Ala-Pro-Phe-p-nitroanilide. The enzymatic properties of mature S188C-subtilisin with the two substrates are almost identical (data not shown). The effect of CLIC and mature S188C-subtilisin on BSA, a large protein substrate, are shown in FIG. 3C. Mature S188C-subtilisin degrades 95% of BSA within 48 hours while CLIC is unable to degrade BSA under the identical conditions. This suggests that CLIC does not have endopeptidase activity against large peptide substrates. While activation of CLIC by the peptide substrate is dependent on both its sequence and concentration (FIG. 3B), large protein substrates like BSA can neither activate CLIC (FIG. 3C) nor be degraded by A-CLIC (data not shown).

Model for Deptide Induced Activation of CLIC

The results indicate that the IMC domain can interact with subtilisin to initiate folding in a manner different from that observed in the IMC-subtilisin complex (Gallagher, T. et al, supra; Jain, S. C. et al., supra). Also, the results are consistent with the second site suppressor mutation that was isolated through a genetic screening method (Kobayashi, T. et al., supra). Second site suppressor mutations in proteins represent a class of amino acid substitutions that when in the presence of the primary mutation can modify the original phenotype of the protein enough to be scored as the wild type. However, since the crosslinking efficiency of the 2Cys-IMC-subtilisin is ˜50%, it appears that alternative pathways for IMC-mediated folding of subtilisin may exist. A possible reason why CLIC displays no endopeptidase activity towards large protein substrates is that the IMC domain functions as a “lid” that sterically occludes bulky substrates. Proteolysis by subtilisin involves: (i) interactions of the substrate with the substrate binding loop in subtilisin (FIG. 1A) and (ii) its subsequent cleavage mediated by the catalytic triad. The binding site and active site, although distinct, are located close to each other (FIGS. 1A, 3D). Although it is presently unknown whether these two sites are completely formed, one may speculate that the lag phase for activation may arise due to the time to form the substrate binding loop or the active site within CLINC. One possible mechanism is that the active site is formed early during folding and the C-terminal region of the IMC domain interacts with and facilitates formation of the substrate binding loop when the IMC is located on the side of the protease domain (FIG. 1A). In CLIC, the C-terminal region of the IMC domain may, however, not be available to form the β-sheet (FIG. 3D), probably due to conformational restrictions imposed by the disulfide bond. When a small peptide is added to CLIC, the peptide substrate may mimic the role of the C-terminal region, albeit with significantly lower efficacy. This may result in several concerted local conformational changes (not detectable by CD) within CLIC to affect its activation. Once the substrate binding loop is formed in CLIC, the lag phase is no longer existent since subsequent substrate molecules bind directly to the substrate binding loop and are degraded by the active site.

The finding that a stably trapped crosslinked conformer can acquire enzymatic activity in the presence of a small peptide substrate suggests that protein folding intermediates of enzymes may have catalytic properties that are different from the native state of the protein. While the formation of such enzymatically active intermediates may be transient, they may still have significance in cells or organisms by serving as spatial and temporal controls.

All of the above-cited publications and patent application are hereby incorporated by reference, as though set forth herein in full.

Those skilled in the art will recognize, or be able to ascertain using no more than routine experimentation, numerous equivalents to the specific biologically active protein folding intermediates, methods and assays described herein. Such equivalents are considered to be within the scope of this invention and are covered by the following claims.

We claim:

1. A method for preparing a biologically active intermediate of a folding protein comprising contacting an intermediate of a folding protein with an effective amount of a substrate or reducing agent.

2. The method of claim 1, wherein the folding protein is subtilisin, α-lytic protease or carboxypeptidase Y.

3. The method of claim 2, wherein the folding protein is subtilisin.

4. The method of claim 3, wherein the substrate is N-succinyl-Ala-Ala-Pro-Phe-p-nitroanilide or N-succinyl-Ala-Ala-Pro-Leu-p-nitroanilide.

5. A biologically active intermediate prepared by contacting an intermediate of a folding protein with an effective amount of a substrate or reducing agent.

6. The intermediate of claim 5, wherein the folding protein is subtilisin, α-lytic protease or carboxypeptidase Y.

7. The intermediate of claim 6, wherein the folding protein is subtilisin.

8. The intermediate of claim 7, wherein the substrate is N-succinyl-Ala-Ala-Pro-Phe-p-nitroanilide or N-succinyl-Ala-Ala-Pro-Leu-p-nitroanilide.

9. A drug design assay comprising assessing a drug candidate's ability to stabilize or prevent formation of a biologically active intermediate of a folding protein.

10. The assay of claim 9, wherein the biologically active intermediate is prepared by contacting an intermediate of a folding protein with an effective amount of a substrate or reducing agent.

11. The intermediate of claim 10, wherein the folding protein is subtilisin, α-lytic protease or carboxypeptidase Y.

12. The intermediate of claim 11, wherein the folding protein is subtilisin.

13. The intermediate of claim 12, wherein the substrate is N-succinyl-Ala-Ala-Pro-Phe-p-nitroanilide or N-succinyl-Ala-Ala-Pro-Leu-p-nitroanilide.
